(19) United States
(12) Patent Application Publication
(10) Pub. No.: US 2014/0309102 A1
(43) Pub. Date: Oct. 16, 2014

(54) Water Gas Shift Catalyst Operating At Medium Temperature And A Process For Its Preparation

(71) Applicant: L'Air Liquide, Societe Anonyme Pour l'Etude et l'Exploitation des Procedes Georges Claude, Paris (FR)

(72) Inventors: Francesco Basile, Catanzaro (IT); Guiseppe Brenna, Rionero in Vulture (IT); Raphael Faure, Villebon-Sur-Yvette (FR); Guiseppe Fornasari, Bologna (IT); Daniel Gary, Montigny le Bretonneaux (FR); Angelo Vaccari, Bologna (IT)

(21) Appl. No.: 14/361,804

(22) PCT Filed: Nov. 14, 2012
(86) PCT No.: PCT/EP2012/072595

§ 371 Date: May 30, 2014

(30) Foreign Application Priority Data
Dec. 2, 2011 (EP) 11306604.7

(51) Int. Cl. B01J 27/236; C07C 29/154; C01B 3/40; B01J 23/80

(52) U.S. Cl. CPC B01J 27/236; B01J 23/80; C07C 29/154; C01B 3/40
USPC 502/176; 502/244; 518/713; 252/373

(57) ABSTRACT

A hydrotalcite-type compound of the formula (I):

[CuₓZn_{y}Al_{w}(OH)₂]^{(2x+2y+3w-2)+}(A²⁻)_{(2x+2y+3w-2)/n},kH₂O (I)
wherein (A²⁻) represents either a carbonate anion or a silicate anion, x>0, y>0, w>0, (x+y)=(1−w), 1<[(x+y)/w]<5, and 1/99≦x/y≦1/1; a synthesis process for its preparation; a catalyst obtained by its calcination and the subsequent reduction of the calcined product.

### CROSS REFERENCE TO RELATED APPLICATIONS

This application is a §371 of International PCT Application PCT/EP2012/072595, filed Nov. 14, 2012, which claims the benefit of EP 11306604.7, filed Dec. 2, 2011, both of which are herein incorporated by reference in their entireties.

### TECHNICAL FIELD OF THE INVENTION

The invention relates to synthesis gas productions and more particularly to a water gas catalyst which is able to be operated at medium temperatures and a process for its preparation.

### BACKGROUND

The water gas shift (WGS) reaction plays a key role in the steam reforming (SR) plants, increasing the hydrogen production and reducing the carbon monoxide content in the exit stream [M. V. Twigg (Ed.), Catalyst Handbook, 2^{nd} ed., Wolfe, London (UK), 1989].

This reaction is currently performed in two steps: a higher temperature step (>350° C.) at the exit of the steam reformer involving iron-based catalysts stabilized by chromium dioxide, and a low temperature step (at about 250° C.) involving copper-based catalysts [K. Klier, “Advances in Catalysis” (D. D. Eley, H. Pines and P. B. Weisz, Eds), Vol. 31, Academic Press, New York, 1982, p. 243; P. Courty and C. Marcilly, “Preparation of Catalysts III” (G. Poncelet, P. Grange and P. A. Jacobs, Eds), Elsevier, Amsterdam (NL), 1983, p. 485]. In the former step, the main objective is to achieve a very low selectivity in the methanation reaction, which is thermodynamically favoured, since the low catalyst activity is compensated both by the temperature and by high carbon monoxide (CO) content. In the latter case, the key objective is to finalize the carbon monoxide conversion, the methanation reaction being significantly reduced at the low temperature.

Hydrotalcite-type (HT) anionic clays have been widely employed as catalysts or catalyst support, on account of the specific and interesting properties of the phases which are obtained by the calcination of the said HT anionic clays, such as a high surface area and homogeneity, a high thermal stability also after reduction, etc.). In particular, Copper-based catalysts obtained from Hydrotalcite-type precursors have been widely used in the synthesis of methanol or of higher molecular weight (HMW) alcohols [P. Courty and C. Marcilly, “Preparation of Catalysts III” (G. Poncelet, P. Grange and P. A. Jacobs, Eds), Elsevier, Amsterdam (NL), 1983, p. 485; F. Cavani et al., Catal. Today 11 (1991), 173; F. Basile and A. Vaccari, “Layered Double Hydroxides—Present and Future” (V. Rives, Ed.), Nova Science, New York (USA), 2001, p. 285; S. Velu et al., Catal. Letters 62 (1999), 159; S. Velu et al, Chem. Commun. (1999), 2341; S. Velu et al. Appl. Catal. A213 (2001), 47; G. Formasari et al., “Catalysis and Automotive Pollution Control” (A. Cruq and A. Frennet, Eds.), Elsevier, Amsterdam (NL), 1987, 469; A.-M. Hilmen, Appl. Catal. 169 (1998), 355; K. J. Smith and R. B. Anderson, Can. J. Chem. Eng. 61 (1983) 40].

The use of Cu-containing catalysts obtained from Hydrotalcite-type precursors in the WGS reaction performed at low temperature has been also widely reported in the patent literature [U.S. Pat. No. 4,835,132; WO 2003/082,468 A1; US 2010/0,102,278 A1, US 2010/0,112,397 A1], evidencing some common features, such as high amounts of copper, to improve the activity, and high amount of aluminium, frequently present and/or added as side phases, to improve the stability with time on stream. For this latter purpose also lanthanum, cerium or zirconium were added. The use of a combination of zinc aluminate and zinc oxide, obtained by calcination of a Zn/Al HT precursor, after doping with significant amounts of alkali (Na, K or Cs), in the water gas shift reaction, is disclosed in EP 2,141,118 A1.

### SUMMARY OF THE INVENTION

The inventors thus developed an original catalyst, which exhibits a superior activity in terms of carbon monoxide conversion, hydrogen formation H₂ yield and carbon dioxide selectivity in medium temperature shift (MTS) operating conditions, without forming of by-products. Moreover said catalyst also exhibits a very good stability upon time-on-stream.

The present invention concerns a new catalyst active in medium temperature shift conditions having a formulation based on an Hydrotalcite-type (HT) precursor containing copper and either carbonate, or silicate as interlayer anions and having a specific phase distribution after calcination and a good Cu dispersion without sintering phenomenon by appropriate temperature reduction treatment. The catalyst according to the invention is obtained by calcination and reduction of Cu/Zn/Al Hydrotalcite-type precursors synthesized by co-precipitation method, with high Cu dispersion.

The catalysts are active and selective in medium temperature shift (MTS) reaction (at about 300° C.), with selectivity in CO₂ and H₂ close to 100%, i.e. without by-products such as methanol or other oxygenate compounds. The catalysts are active and stable between 250° C. and 350° C. and operating at low residence times, reaching the equilibrium values already at 300° C. with a residence time of only 1.0 sec.

The invention also relates to the possibility to obtain high activity, selectivity and stability of Cu-containing catalysts obtained by reduction of specific mixed oxides in which the Cu²⁺ ion is present after calcination of the precursor. Layered hydrotalcite-type anionic clays have been used as precursors to obtain new catalysts, with unusual properties due to the presence of all active elements well dispersed inside brucite-like layers of the precursors. Hydrotalcite-type phases form, by controlled calcination, mixed oxides with high thermal stability, surface area and active-phase dispersion, factors directly affecting the catalytic activity. Hydrotalcite-type precursors are prepared by co-precipitation of all the elements to obtain homogeneous precipitates.

The study refers to stable formulations that not only may exhibit good physicochemical properties in the medium temperature shift conditions, but also may act as active phase in the water gas shift reaction.

According to a first embodiment, the invention relates to an hydrotalcite-type compound of the formula (I):

[CuₓZn_{y}Al_{w}(OH)₂]^{(2x+2y+3w-2)+}(A²⁻)_{(2x+2y+3w-2)/2},kH₂O (I)

Wherein:
(A²⁻) represents either a carbonate anion or a silicate anion,
x>0,
y>0,
w>0,
(x+y)=(1−w),
1<[(x+y)/w]<5, and
1/99≦x/y≦1/1.

According to a particular embodiment, the atomic ratio x/y≦1/2, in the formula (I) as hereinbefore defined.

According to a particular embodiment, the atomic ratio x/y≦1/5, in the formula (I) as hereinbefore defined

According to another particular embodiment, the atomic ratio x/y≧1/10, in the formula (I), as hereinbefore defined.

According to another particular embodiment, the atomic ratio [(x+y)/w] is higher or equal to 2, in the formula (I), as hereinbefore defined.

According to another particular embodiment, the atomic ratio [(x+y)/w] is less or equal to 3, in the formula (I), as hereinbefore defined.

According to a more particular embodiment, the hydrotalcite-type compound of the formula (I), as hereinbefore defined is of one of the following formulas:

[Cu_{0.075}Zn_{0.675}Al_{0.25}(OH)₂]^{0.25+}(CO₃²⁻)_{0.125}kH₂O,

[Cu_{0.150}Zn_{0.600}Al_{0.25}(OH)₂]^{0.25+}(CO₃²⁻)_{0.125}kH₂O,

[Cu_{0.066}Zn_{0.600}Al_{0.333}(OH)₂]^{0.25+}(CO₃²⁻)_{0.125}kH₂O,

[Cu_{0.134}Zn_{0.532}Al_{0.333}(OH)₂]^{0.31+}(CO₃²⁻)_{0.155}kH₂O.

According to another embodiment, the invention relates to a synthesis process of the Hydrotalcite-type compound of the formula (I) as hereinbefore defined, comprising the following steps:
A step A during which an aqueous solution containing together, aluminium nitrate, zinc nitrate and copper nitrate is prepared by mixing said nitrate salts in the desired molar proportions, with water;
A step B during which, said solution obtained at step A is mixed with an aqueous solution of sodium carbonate, the pH being maintained between 8 and 10, preferably around 9, to produce a precipitate;
A step C during which, said precipitate obtained at step B, is isolated by filtration, washed and then dried to form the expected hydrotalcite-type compound of said formula (I).

According to a particular embodiment, the synthesis process as hereinabove defined further comprises:
A step D during which said hydrotalcite-type compound of said formula (I) as hereinabove defined, obtained at step C, is grinded to form a powder of particles of said hydrotalcite-type compound of said formula (I).

According to another embodiment, the invention relates to a compound characterized in that it is obtained by a process comprising the following steps:
A step F during which the powder of the hydrotalcite-type compound of the formula (I) as hereinbefore defined is calcined;
A step G during which the calcined powder obtained at step F is reduced with hydrogen at a temperature of less than 230° C.

According to another embodiment, the invention relates to the use of the compound obtained by the process as hereinabove defined, as a catalyst for the water gas shift reaction of synthesis gas or as a catalyst for the synthesis of methanol by hydrogenation of CO or CO₂.

### BRIEF DESCRIPTION OF THE DRAWINGS

These and other features, aspects, and advantages of the present invention will become better understood with regard to the following description, claims, and accompanying drawings. It is to be noted, however, that the drawings illustrate only several embodiments of the invention and are therefore not to be considered limiting of the invention's scope as it can admit to other equally effective embodiments.

FIG. 1 provides graphical data

FIG. 2 provides graphical data.

FIG. 3 provides graphical data.

FIG. 4 provides graphical data.

FIG. 5 provides graphical data.

FIG. 6 provides graphical data.

FIG. 7 provides graphical data.

FIG. 8 provides graphical data.

### DETAILED DESCRIPTION

The following paragraphs disclose non-limitative examples of catalysts according to the present invention.

A] Synthesis of Catalysts

### Example 1

### Preparation of Catalyst ZAC1c

This example illustrates the preparation process of 20 grams of catalyst containing 10 wt. % of copper and having an atomic ratio (Cu+Zn)/Al=3, using a carbonate as an intercalated anion in the hydrotalcite-type precursor.

A copper, zinc and aluminium salts dimolar (2M) aqueous solution is prepared from 7.321 g of 99.99% copper nitrate hemipentahydrate [Cu(NO₃)₂, 2.5H₂O], 51.165 g of 98.00% zinc nitrate hexahydrate [Zn(NO₃)₂, 6H₂O] and 26.016 g of 98.00% aluminium nitrate nonahydrate [Al(NO₃)₃, 9H₂O] in about 136.0 cm³ of deionised water.

This solution is then drop by drop poured, under energetic magnetic stirring, into a molar (1M) solution containing 14.479 g of 99.50% of sodium carbonate (Na₂CO₃) in about 136.0 cm³ of deionised water at 60° C., while maintaining the pH to 9.0±0.1, by a drop by drop addition of a trimolar (3M) aqueous solution of sodium hydroxide (NaOH).

The obtained precipitate is aged well dispersed in the same conditions (at 60° C. and pH=9.0) under energetic magnetic stirring for 45 min. The solid precipitate is then separated from the mother liquor by filtration with a Buchner funnel, vacuum being provided by a Venturi water suction device.

The resulting solid corresponds to a hydrotalcite-type compound of the formula:

[Cu_{0.117}Zn_{0.629}Al_{0.254}(OH)₂]^{0.25+}(CO₃²⁻)_{0.125}kH₂O,

This resulting solid is then washed with abundant hot water (60° C., 400 cm³/g of sample) and dried overnight at 70° C. After grinding the obtained precursor, the powder is calcined at 550° C. (10° C./min) in a muffle furnace for 6 h and then formed and sieved with a size from 30 to 40 mesh.

### Example 2

### Preparation of Catalyst ZAC2c

The preparation process is worked as in Example 1 except for the Cu content (wt. %), which is two times more than in Example 1 (ZAC1c).

The metal salts 2M aqueous solution is prepared from 14.641 g of 99.99% [Cu(NO₃)₂, 2.5H₂O], 42.000 g of 98.00% [Zn(NO₃)₂, 6H₂O] and 26.092 g of 98.00% [Al(NO₃)₃, 9H₂O] in about 136.0 cm³ of deionised water.

This solution is then drop by drop poured, under energetic magnetic stirring, into a 1M solution containing 14.521 g of 99.50% of Na₂CO₃ in about 136.0 cm³ of deionised water at 60° C., while maintaining the pH to 9.0±0.1, by a drop by drop addition of a 3M aqueous solution of NaOH.

The obtained precipitate is aged well dispersed in the same conditions (at 60° C. and pH=9.0) under energetic magnetic stirring for 45 min. The solid precipitate is then separated from the mother liquor by filtration with a Buchner funnel, vacuum being provided by a Venturi water suction device.

The resulting solid corresponds to a hydrotalcite-type compound of the formula:

[Cu_{0.234}Zn_{0.514}Al_{0.252}(OH)₂]^{0.25+}(CO₃²⁻)_{0.125}kH₂O,

This resulting solid is washed with abundant hot water (60° C., 400 cm³/g of sample) and dried overnight at 70° C. After grinding the obtained precursor, the powder is calcined at 550° C. (10° C./min) in a muffle furnace for 6 h and then formed and sieved with a size from 30 to 40 mesh.

### Example 3

### Preparation of Catalyst ZAC3c

The preparation process is worked as in Example 1 except for the Cu content (wt. %), which is three times more than in Example 1 (ZAC1c).

The metal salts 2M aqueous solution is prepared from 21.962 g of 99.99% [Cu(NO₃)₂, 2.5H₂O], 32.837 g of 98.00% [Zn(NO₃)₂, 6H₂O] and 26.167 g of 98.00% [Al(NO₃)₃, 9H₂O] in about 137.0 cm³ of deionised water.

This solution is then drop by drop poured, under energetic magnetic stirring, into a 1M solution containing 14.563 g of 99.50% of Na₂CO₃ in about 136.0 cm³ of deionised water at 60° C., while maintaining the pH to 9.0±0.1, by a drop by drop addition of a 3M aqueous solution of NaOH.

The obtained precipitate is aged well dispersed in the same conditions (at 60° C. and pH=9.0) under energetic magnetic stirring for 45 min. The solid precipitate is then separated from the mother liquor by filtration with a Buchner funnel, vacuum being provided by a Venturi water suction device.

The resulting solid correspond to a hydrotalcite-type compound of the formula:

[Cu_{0.349}Zn_{0.399}Al_{0.251}(OH)₂]^{0.25+}(CO₃²⁻)_{0.125}kH₂O,

The resulting solid is washed with abundant hot water (60° C., 400 cm³/g of sample) and dried overnight at 70° C. After grinding the obtained precursor, the powder is calcined at 550° C. (10° C./min) in a muffle furnace for 6 h and then formed and sieved with a size from 30 to 40 mesh.

### Example 4

### Preparation of Catalyst ZAC1s

The preparation process is worked as in Example 1, except that the Na₂CO₃ solution being replaced by a sodium silicate (Na₂SiO₃) solution.

The metal salts 2M aqueous solution is prepared from 7.321 g of 99.99% [2Cu(NO₃)₂, 5H₂O], 51.165 g of 98.00% [Zn(NO₃)₂, 6H₂O] and 26.016 g of 98.00% [Al(NO₃)₃, 9H₂O] in about 136.0 cm³ of deionised water.

This solution is then drop by drop poured, under energetic magnetic stirring, into a 1M aqueous solution of sodium silicate (Na₂SiO₃) prepared from 9.103 g of sodium tri-silicate solution (27.0% SiO₂) in 41.0 cm³ of deionised water, at 60° C., while maintaining the pH to 9.0±0.1, by a drop by drop addition of a 3M aqueous solution of NaOH.

The obtained precipitate is aged well dispersed in the same conditions (at 60° C. and pH=9.0) under energetic magnetic stirring for 45 min. The solid precipitate is then separated from the mother liquor by filtration with a Buchner funnel, vacuum being provided by a Venturi water suction device.

The resulting solid correspond to a hydrotalcite-type compound of the formula:

[Cu_{0.117}Zn_{0.629}Al_{0.254}(OH)₂]^{0.25+}(SiO₃²⁻)_{0.125}kH₂O,

This resulting solid is washed with abundant hot water (60° C., 400 cm³/g of sample) and dried overnight at 70° C. After grinding the obtained precursor, the powder is calcined at 550° C. (10° C./min) in a muffle furnace for 6 h and then formed and sieved with a size from 30 to 40 mesh.

### Example 5

### Preparation of Catalyst ZAC2s

The process of Example 2 is followed except that the carbonate solution is replaced by the silicate one.

The metal salts (2M) aqueous solution is prepared from 21.962 g of 99.99% [2Cu(NO₃)₂, 5H₂O], 32.837 g of 98.00% [Zn(NO₃)₂, 6H₂O] and 26.167 g of 98.00% [Al(NO₃)₃, 9H₂O] in about 137.0 cm³ of deionised water.

This solution is then drop by drop poured, under energetic magnetic stirring, into a 1M aqueous solution of Na₂SiO₃ prepared from 9.077 g of sodium tri-silicate solution (27.0% SiO₂) in 41.0 cm³ of deionised water, at 60° C., while maintaining the pH to 9.0±0.1, by a drop by drop addition of a 3M aqueous solution of NaOH.

The obtained precipitate is aged well dispersed in the same conditions (at 60° C. and pH=9.0) under energetic magnetic stirring for 45 min. The solid precipitate is then separated from the mother liquor by filtration with a Buchner funnel, vacuum being provided by a Venturi water suction device.

The resulting solid corresponds to a hydrotalcite-type compound of the formula:

[Cu_{0.234}Zn_{0.514}Al_{0.252}(OH)₂]^{0.25+}(SiO₃²⁻)_{0.125}kH₂O,

This resulting solid is washed with abundant hot water (60° C., 400 cm³/g of sample) and dried overnight at 70° C. After grinding the obtained precursor, the powder is calcined at 550° C. (10° C./min) in a muffle furnace for 6 h and then formed and sieved with a size from 30 to 40 mesh.

### Example 6

### Preparation of Catalyst ZAC1cK

The preparation process is worked as in Example 1; however, after the calcination a potassium carbonate (2 wt. % K) (K₂CO₃) solution is used to dope the catalyst by Incipient Wetness Impregnation (IWI) method.

A copper, zinc and aluminium salts (2M) aqueous solution is prepared from 7.321 g of 99.99% [2Cu(NO₃)₂, 5H₂O], 51.165 g of 98.00% [Zn(NO₃)₂, 6H₂O] and 26.016 g of 98.00% [Al(NO₃)₃, 9H₂O] in about 136.0 cm³ of deionised water.

This solution is then drop by drop poured, under energetic magnetic stirring, into a 1M aqueous solution containing 14.479 g of 99.50% Na₂CO₃ in about 136.0 cm³ of deionised water at 60° C., while maintaining the pH to 9.0±0.1, by a drop by drop addition of a 3M aqueous solution of NaOH.

The obtained precipitate is aged well dispersed in the same conditions (at 60° C. and pH=9.0) under energetic magnetic stirring for 45 min. The solid precipitate is then separated from the mother liquor by filtration with a Buchner funnel, vacuum being provided by a Venturi water suction device.

The resulting solid is washed with abundant hot water (60° C., 400 cm³/g of sample) and dried overnight at 70° C. After grinding the obtained precursor, the powder is calcined at 550° C. (10° C./min) in a muffle furnace for 6 h and then formed and sieved with a size from 30 to 40 mesh.

A potassium solution is prepared from 0.31 g of K₂CO₃ in 25 cm³ of deionised water.

The calcined sample is then impregnated with this potassium solution by the IWI technique, dried again at 120° C. for 2 h and, then calcined at 550° C. (10°/min) in a muffle furnace for 2 h.

### Comparative Example

### Commercial MTS Catalyst

The comparative example is a commercial Cu-based catalyst, optimized to perform the water gas shift reaction at low and/or at medium temperature.

B] Catalysts Characterization

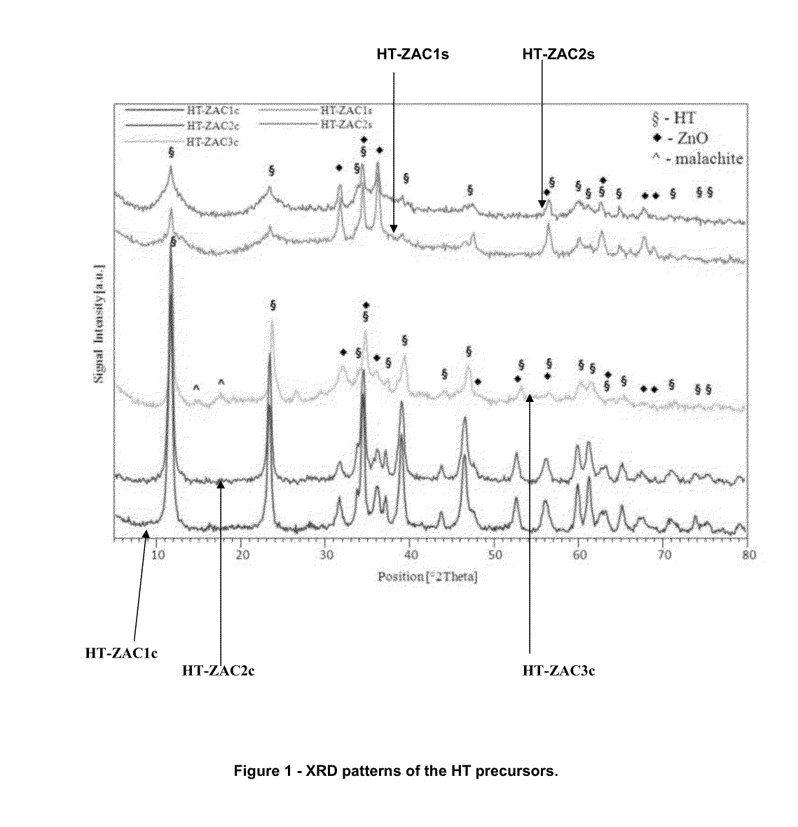

FIG. 1 shows the graphs resulting from the XRD analysis of each of the hydrotalcite-type precursors of catalysts ZAC1c, ZAC2c, ZAC3c, ZAC1s and ZAC2s (identified as HT-ZAC1c, HT-ZAC2c, HT-ZAC3c, HT-ZAC1s and HT-ZAC2s).

The presence of carbonate or silicate ions, in particular the first one, during precipitation results in the HT-structure formation, as identified by XRD analysis. The curve of HT-ZAC3c also has small reflections which can be attributed to a malachite-like phase.

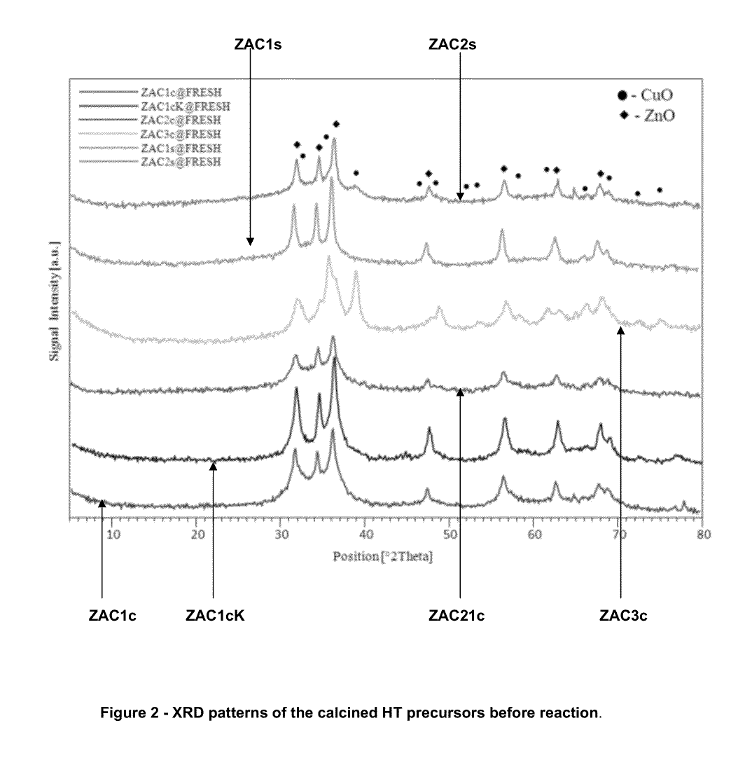

As shown on the graphs of FIG. 2, the HT-structure containing carbonate ions topotactically evolves after calcination, and XRD shows only a ZnO-like phase without any segregation of Cu-containing species, except for the sample with the highest amount of copper (ZAC3c), which shows also the CuO phase. On the contrary ZnO-like phase is the main phase when silicate ion is used as intercalated anion in the HT-precursor preparation, both before and after calcination.

A summary of the chemical-physical properties is reported in Tables 1a and 1b.

The ZAC1cK sample does not show any differences, before and after reaction, in terms of both XRD phases and BET surface values. In the other cases, the effect of sintering is confirmed by the surface area values, which decrease after reaction almost for all samples and is more evident in ZAC1c and ZAC1s samples (the lowest Cu content).

TABLE 1a

| | | | S_{BET} | MSA^{b} | S_{Cu}^{b} |
|---|---|---|---|---|---|
| Sample | | Phases | [m²/g] | [m²/g_{CAT}] | [m²/g_{Cu}] |
| ZAC1c | Fresh | ZnO | 62 | 3.5 | 35 |
| | Used | Cu⁰ (tr), ZnO, | 34 | 3.1 | 31 |
| | | spinel | | | |
| ZAC1cK | Fresh | ZnO | 50 | 2.7 | 27 |
| | Used | CuO, ZnO, spinel | 34 | 2.1 | 21 |
| ZAC2c | Fresh | ZnO | 48 | 4.9 | 25 |
| | Used | Cu⁰, ZnO, spinel | 34 | 5.1 | 25 |
| ZAC3c | Fresh | CuO, ZnO | 22 | 3.8 | 13 |
| | used | Cu⁰, CuO, ZnO, | 18 | 3.9 | 13 |
| | | spinel (tr) | | | |
| ZAC1s | Fresh | CuO, ZnO | 129 | 4.7 | 47 |
| | Used | CuO, ZnO, spinel | 63 | 3.7 | 37 |
| | | (tr) | | | |
| ZAC2s | Fresh | CuO, ZnO | 110 | 7.1 | 35 |
| | Used | CuO, ZnO, spinel | 110 | 6.8 | 34 |
| | | (tr) | | | |

TABLE 1b

| | | | d_{Cu}* | d_{ZnO}^{a} | D^{b} |
|---|---|---|---|---|---|
| Sample | | Phases | [nm] | [nm] | [%] |
| ZAC1c | Fresh | ZnO | 19^{b} | 15 | 5.4 |
| | Used | Cu⁰ (tr), ZnO, | 21^{b} | 20 | 4.8 |
| | | spinel | | | |
| ZAC1cK | Fresh | ZnO | 25^{b} | 21 | 4.1 |
| | Used | CuO, ZnO, spinel | 32^{b} | 35 | 3.3 |
| ZAC2c | Fresh | ZnO | 27^{b} | 15 | 3.8 |
| | Used | Cu⁰, ZnO, | 12^{a} (Cu), 27^{b} | 16 | 3.5 |
| | | spinel | | | |
| ZAC3c | Fresh | CuO, ZnO | 20^{a} (CuO), 51^{b} | 20 | 2.0 |
| | used | Cu⁰, CuO, ZnO, | 14^{b} | 32 | 2.0 |
| | | spinel (tr) | | | |
| ZAC1s | Fresh | CuO, ZnO | 18^{b} | 28 | 7.2 |
| | Used | CuO, ZnO, spinel | 63 | 20 | 5.7 |
| | | (tr) | | | |
| ZAC2s | Fresh | CuO, ZnO | 19^{b} | 21 | 5.4 |
| | Used | CuO, ZnO, spinel | 16^{a} (CuO), 20^{b} | 6.8 | 5.3 |
| | | (tr) | | | |

^{a}= measured by Debey-Scherrer formula;
^{b}= measured by N₂O chemisorption;
*= CuO (before reaction); CuO and Cu⁰ (after reaction)

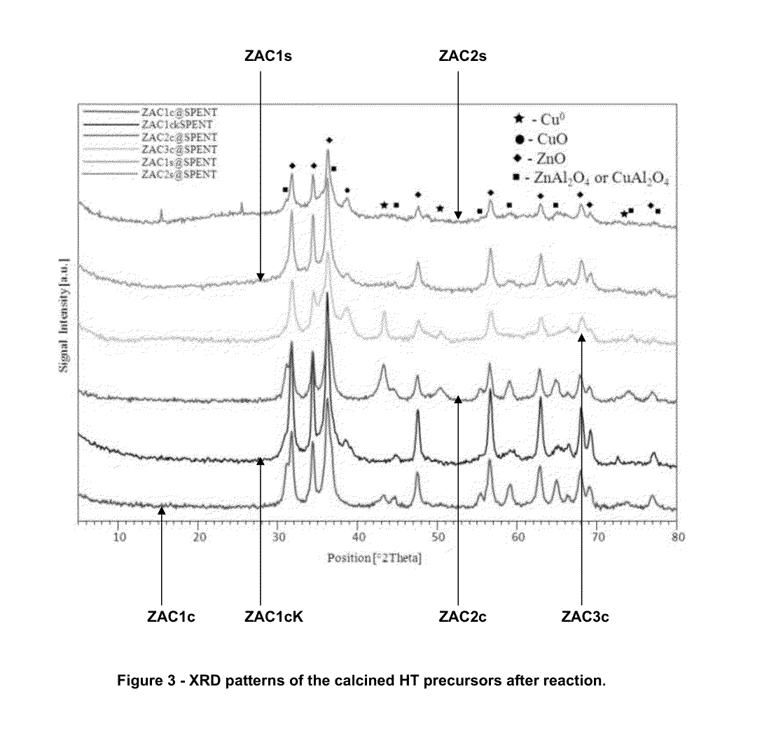

As shown on the graphs of FIG. 3, the characteristic reflections of Cu⁰ at 43.3, 50.5 and 74.1° are observed in the Cu-containing samples, confirming the catalyst activation. It happens mainly by increasing the content of Cu or after reaction.

The crystal size of the Cu-containing samples, before and after reaction, are calculated by the Debey-Scherrer formula [H. P. Klug and L. E. Alexander, X-Ray Diffraction Procedures, Wiley, New York (USA), 1974], by using the best resolved reflections: at 36.2° for ZnO, 38.9° for CuO and at 43.3° for Cu⁰.

The samples show an increase of crystal size after reaction, evidencing a slight structural sintering of copper and support. A sintering effect is evident in the Cu-containing samples after reaction, by increasing the Cu content.

The pore size distributions (3-40 nm) and the isotherms, grouped into TYPE IV (hysteresis loop of TYPE H4) of the IUPAC classification [IUPAC. Pure Appl. Chem. 57 (4) (1985), 603], indicate that the calcined HT catalysts contain mainly mesopores.

The HT precursors after calcination have been reduced before the catalytic tests under MTS conditions, to obtain the main active phase. During the reduction step, the hot spot temperature is controlled at 220° C. and never should be allowed to exceed 230° C. The N₂ pressure is 10⁶ Pa (lobar) with a gas hourly space velocity (GHSV) between 300 h⁻¹ and 400 h⁻¹.

A typical procedure comprises:
1) To remove oxygen (O₂) by purging nitrogen (N₂) in the reactor and, after that, to heat the catalyst to 175° C. (50° C./h);
2) To add a flow of 0.8 v/v % of hydrogen (H₂), when the temperature of 175° C. is reached and kept constant;
3) To increase step by step the H₂ concentration with step of 0.2 v/v % without exceeding 1.2 v/v % of H₂/N₂ at this stage for 18 h;
4) To increase the inlet temperature up to 220° C. (15° C./h), avoiding to exceed 1.5 v/v % of H₂ in N₂;
5) To increase step by step the H₂ concentration up to 4 v/v % in N₂, with step of 0.5 v/v % and, when the temperature of 220° C. is reached and stabilized, to check that the catalyst hot spot temperature does not exceed 230° C. If the catalyst hot spot exceeds 250° C., the protocol must be interrupted (injection of H₂/N₂ stopped, depressurization of the reactor and sweeping by fresh N₂).
6) The reduction is considered complete when a H₂ consumption of less than 0.2 v/v % is consecutively assessed during 2 h.

For the Cu-containing samples, it is possible to hypothesize the presence of different Cu-containing species on the basis of the literature data [J. D. Stroupe, J. Am. Chem. Soc. 71 (1949), 569], with different oxide-oxide surface interactions. The H₂-TPR profiles of all calcined ZAC catalysts show that, before and after reaction, they are completely reduced at temperatures below 300° C. with a peak typical of the reduction of the highly dispersed Cu species [W. R. A. M. Robinson and J. C. Mol, Appl. Catal. 60 (1990), 61; G. L. Castiglioni, Appl. Catal. 123 (1995), 123; J. Als-Nielsen et al., Nucl. Instrum. Methods Phys. Res. B97 (1995), 522; T. L. Reitz et al., J. Catal. 199 (2001), 193].

ZnO-like phase does not reduce under the experimental conditions, in agreement to the literature [Y. Okamoto et al., J. Phys. Chem. 87 (1983), 3740.], such as confirmed by the ZA3K sample that did not reduce under the analysis conditions.

C1] Catalytic Activity Test 1

The catalysts of the present invention together with the reference catalyst (ZA3K) are shaped as pellets with size between 30 and 40 mesh and tested in a plug-flow reactor.

The tubular reactor is heated by an oven in order to have a temperature between 250° C. and 350° C. (±1° C.), measured immediately at the exit of the catalytic bed, and pressurized under 15×10⁵ Pa (15 bar).

Dry Gas (DG) and Steam (S) are pre-heated at around 215° C. and mixed (mass flow controller) before passing over the catalyst. In order to determine the activity of the catalysts produced by the various examples, in medium temperature shift (MTS) operating conditions, using a typical DG composition containing 18.8 vol % carbon monoxide, 4.6 vol % carbon dioxide, 4.6 vol % methane and hydrogen up to 100 vol %. This gas flow and steam are passed over the pre-reduced catalysts with a steam to dry gas v/v ratio (S/DG) of 0.55 and 0.25. Concentration of all components is regularly measured both inlet and exit dry gas by means of Perkin Elmer gas chromatograph calibrated towards a gas mixture of known composition. The Gas Hourly Space Velocity (GHSV) is between 3,600 and 14,400 h⁻¹.

It was firstly checked if a Zn/Al-based catalyst, according to EP 2,141,118, shows a significant activity in MTS operating conditions. This Zn/Al calcined HT precursor has a molar ratio equal to 3 and is doped with 2 wt. % K, (reported in the text as “ZA3K”).

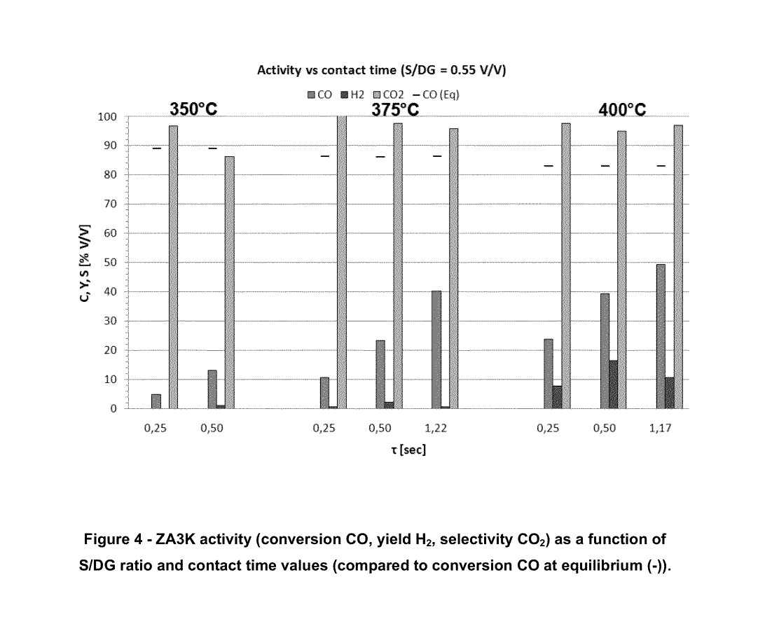

As shown on FIG. 4, the 2 wt. % K-doped (ZA3K) sample is active at temperature higher than 350° C. with a CO conversion that reaches 40% in condition of larger excess of steam (S/DG=0.55 v/v).

Although the operating conditions are favourable, H₂ yield is always lower than the CO conversion value and reaches the highest value at 400° C., which may be explained by the presence of side reactions with H₂ consumption which are favoured by the high surface basic character, with formation of oxygenated product [K. J. Smith and R. B. Anderson, Can. J. Chem. Eng. 61 (1983) 40; Y. Okamoto et al., J. Phys. Chem. 87 (1983), 3740; C. E. Hofstadt et al., In Preparation of Catalysis III (G. Poncelet, P. Grange and P. A. Jacobs, Eds), Elsevier, Amsterdam (NL), 1983.]. This was confirmed by the HPLC analyses on the liquid condensates showing a significant presence of oxygenated products, mainly methanol.

Differently, Cu adding in the Zn/Al mixed oxide gives a real improvement in terms of CO conversion and H₂ yield. All results observed under all the operating conditions are summarized in the Table 2.

The samples obtained from carbonate-containing HT precursors (ZAC1c and ZAC2c) show the best performance in all the temperature range investigated, reaching the equilibrium values of CO conversion already at 300° C. The ZAC2c sample has a Cu-content two times higher that ZAC1c (but remaining lower than 20 wt. %), trying to observe improved results at temperature between 250 and 300° C.

TABLE 2
Summary of all results in terms of CO conversion and H₂ yield.

| | | | | | | | | | | | | |
|---|---|---|---|---|---|---|---|---|---|---|---|---|
| | T (° C.) | 250 | 250 | 250 | 300 | 300 | 300 | 350 | 350 | 350 | 250 | 300 |
| | S/DG (v/v) | 0.55 | 0.55 | 0.25 | 0.55 | 0.55 | 0.25 | 0.55 | 0.55 | 0.25 | 0.55 | 0.55 |
| | τ (s)* | 0.50 | 1.00 | 1.00 | 0.50 | 1.00 | 1.00 | 0.50 | 1.00 | 1.00 | 0.50 | 0.25 |
| ZAC1c | CO conv. (%) | 76.4 | 89.4 | 57.6 | 92.5 | 92.7 | 75.5 | 88.1 | 88.0 | 60.2 | 72.0 | 83.3 |
| | H₂ yield (%) | 75.6 | 88.7 | 54.7 | 90.6 | 91.7 | 72.6 | 85.9 | 86.1 | 5.8 | 70.1 | 82.8 |
| ZAC2c | CO conv. | 85.5 | 92.4 | 68.1 | 91.9 | 92.2 | 76.7 | 87.4 | 86.8 | 69.2 | 78.2 | 81.6 |
| | | 95.8 | 86.3 | 67.0 | 91.1 | 91.6 | 74.2 | 80.0 | 84.4 | 67.2 | 75.6 | 79.5 |
| ZAC3c | CO conv. | 24.1 | 37.3 | 20.7 | 47.5 | 61.8 | 43.0 | 68.8 | 74.5 | 56.9 | 17.4 | 26.8 |
| | | 24.0 | 37.3 | 23.9 | 44.9 | 63.5 | 41.6 | 69.2 | 74.7 | 57.4 | 15.8 | 24.6 |
| ZAC1c | CO conv. | 32.4 | 49.7 | 30.9 | 61.9 | 76.3 | 58.7 | 79.8 | 85.5 | 65.2 | 22.0 | 33.8 |
| K | | 33.5 | 50.1 | 29.4 | 62.3 | 75.8 | 59.6 | 79.6 | 86.5 | 66.2 | 21.8 | 35.7 |
| ZAC1s | CO conv. | 25.3 | 30.7 | 15.2 | 42.8 | 58.6 | 37.4 | 64.6 | 59.5 | 42.0 | 6.4 | 10.0 |
| | | 25.7 | 33.3 | 15.0 | 42.1 | 57.9 | 37.4 | 65.1 | 62.1 | 44.7 | 4.8 | 10.8 |
| ZAC2s | CO conv. | 15.5 | 15.4 | 8.2 | 47.7 | 36.2 | 27.4 | 64.3 | 55.3 | 39.4 | 10.1 | 10.9 |
| | | 15.4 | 15.2 | 8.0 | 46.1 | 34.9 | 22.9 | 66.2 | 54.9 | 39.1 | 11.9 | 11.2 |

* τ (s): Contact time (in seconds)

As expected, the decrease of the S/DG ratio has a negative effect at all the temperatures, although it is significant only at 250° C. where the CO conversion decreases from 89 to 58% (ZAC1c) and from 92 to 68% (ZAC2c) by decreasing the S/DG value from 0.55 to 0.25.

At temperatures above 300° C., the S/DG ratio effect on the activity of ZAC1c and ZAC2c catalysts is negligible, the CO conversion and the H₂ yield values being always close to the equilibrium values. The catalytic performances in the water gas shift reaction are strongly affected by the S/DG ratio for all the other catalysts.

The effect of the contact time (τ) is significant at 350° C. for the carbonate-derived catalysts having a Cu content higher than 20 wt % and for all the silicate-derived catalysts.

Considering that the GHSV is higher than the typical industrial values, the activity of the carbonate-derived catalysts in the WGS reaction is slightly affected by the contact time: at 300° C., because the CO conversion only decreased if the lowest contact time value (0.25 sec) is used, whereas at 250° C. it reaches 90% (ZAC2c) with a contact time of 1.0 sec. At 300° C., only a drastic decrease of contact time causes a decrease in both CO conversion and H₂ yield values.

The selectivity is maximum in the syngas products, while lower by-products formation (i.e. methanol) and no carbon formation have been observed. The H₂ yield has practically the same trend of CO conversion: this result, together with the CO₂ selectivity values (always higher than 97%), allows excluding the presence of significant side-reactions. Therefore, the activity for the tested samples seems to be affected by the thermodynamic (steam excess) and kinetics (contact time τ) parameters, in particular at 250° C. However, these samples are expected to reach the equilibrium value of CO conversion at 250° C. by using the conventional contact time values of industrial plants.

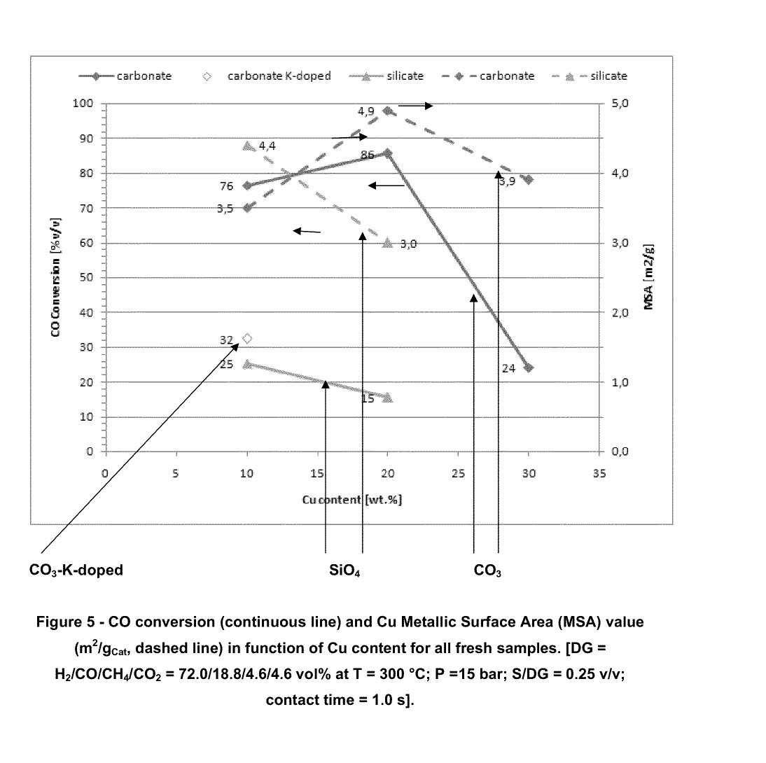

As shown on the graph of FIG. 5, only a rough correlation exists between the Cu⁰-surface area and the catalytic activity (FIG. 5).

For the samples obtained from carbonate-containing HT precursors, after a first slight increase, a dramatic decrease may be observed for the higher Cu-content, although the effect on the metallic surface area (MSA) is significantly lower than that on the catalytic activity.

For the samples obtained from silicate-containing HT-precursors the decrease of CO conversion is significantly higher in comparison to the former samples, than that scheduled on the basis of metallic surface area. This suggests the existence of synergetic effects between Cu- and Zn-containing phases, favoured in by the specific nature of the precursors according to the present invention.

C2] Catalytic Activity Test 2

The activity and selectivity of the ZAC catalyst is compared to those of commercial material used for MeOH synthesis and MTS and LTS reactions. The following table clearly shows the best performance of the Ex-Hydrotalcite Cu-based catalyst derived from the calcination of carbonate-containing precursor, in terms of selectivity and productivity, under temperature conditions, between 280 and 320° C.

TABLE 3
catalysts performances for hydrogenation to MeOH

| | | | | | | | | | | Productivity |
|---|---|---|---|---|---|---|---|---|---|---|
| | | Cu | Cat | | ToS | | | | | MeOH |
| | Density | load | load | Temperature | (duration) | Conversions (%) | | Selectivities (%) | | g_{MeOH}/k |
| Catalyst | (apparent) | W % | (mg) | ° C. | h | H₂ | CO₂ | MeOH | CO | g_{cata}/h |
| ZAC2c | 0.84 | 20 | 202.0 | 280° C. | 22.8 | 0.1 | 0.8 | 15 | 85 | 3 |
| | | | | 300° C. | 88.5 | 3.9 | 7.1 | 52 | 48 | 102 |
| | | | | 320° C. | 20.7 | 7.7 | 14.8 | 48 | 52 | 195 |
| 1 - | 1.41 | 40 | 338.6 | 280° C. | 20.8 | 0.7 | 0.8 | 74 | 26 | 10 |
| MeOH | | | | 300° C. | 65.5 | 3 | 4.7 | 78 | 22 | 61 |
| synthesis | | | | 320° C. | 44.4 | 6.3 | 10.9 | 59 | 41 | 105 |
| 2 - MTS | 1.09 | 15 | 261.8 | 280° C. | 18.1 | 0 | 3 | 3 | 97 | 2 |
| process | | | | 300° C. | 66.2 | 4 | 12 | 25 | 75 | 63 |
| | | | | 320° C. | 42.7 | 8 | 17 | 31 | 69 | 112 |
| 3 - LTS | 1.04 | 40 | 250.6 | 280° C. | 19.7 | 0 | 0 | 95 | 5 | 6 |
| process | | | | 300° C. | 48.8 | 3 | 3 | 87 | 13 | 66 |
| | | | | 320° C. | 65.1 | 4 | 6 | 79 | 21 | 112 |
| 4 - LTS | 1.33 | 40 | 319.7 | 280° C. | 18.6 | 7 | 14 | 41 | 59 | 98 |
| process | | | | 300° C. | 46.8 | 10 | 23 | 37 | 63 | 147 |
| | | | | 320° C. | 65.1 | 11 | 26 | 33 | 67 | 151 |

1) 2) 3) 4) catalysts are commercial samples. Catalyst 1) is used for MeOH synthesis from syngas mixture and catalysts 2) 3) 4) used for MTS or LTS processes

In conclusion, the ZAC catalyst is highly active and selective towards MeOH productivity, this latter close to that of the best commercial LTS catalyst having 2.0 times more Cu. ZAC catalyst performances are higher than that of a commercial catalyst designed for conventional MeOH synthesis route.

The very good performances of ZAC2c for these operating conditions, associated with a good thermal stability, justify the use of ZAC2c catalysts in the hydrogenation of CO₂ processes. The low amount of Cu is justified in order to minimize sintering phenomena (potential risk due to the elevated range of temperature (i.e. 320° C.).

The innovative catalyst (ZAC) allows to the synthesis of MeOH by hydrogenation of CO₂ using severe operating conditions (320° C. instead of current 230° C.).

For same range of pressure and GHSV, the MeOH yield is improved significantly vs those obtained by means of suitable commercial products.

Production of by-product is negligible (not detected).

D] Catalyst Stability

The possible deactivation of the catalysts is studied at 300° C. for a period of 100 h, by using a most favourable contact time (1.0 sec), but in hard conditions in term of S/DG value (0.25 v/v). All the prepared catalysts show optimal stability with Time-on-Stream (tos), particularly the catalysts derived from the calcination of carbonate HT precursors. The same behaviour in terms of stability is observed for all the investigated samples, (Examples 1 to 6). As the equilibrium values are reached in more soft conditions, these results suggest a good performance of the catalysts in the middle temperature shift operating conditions.

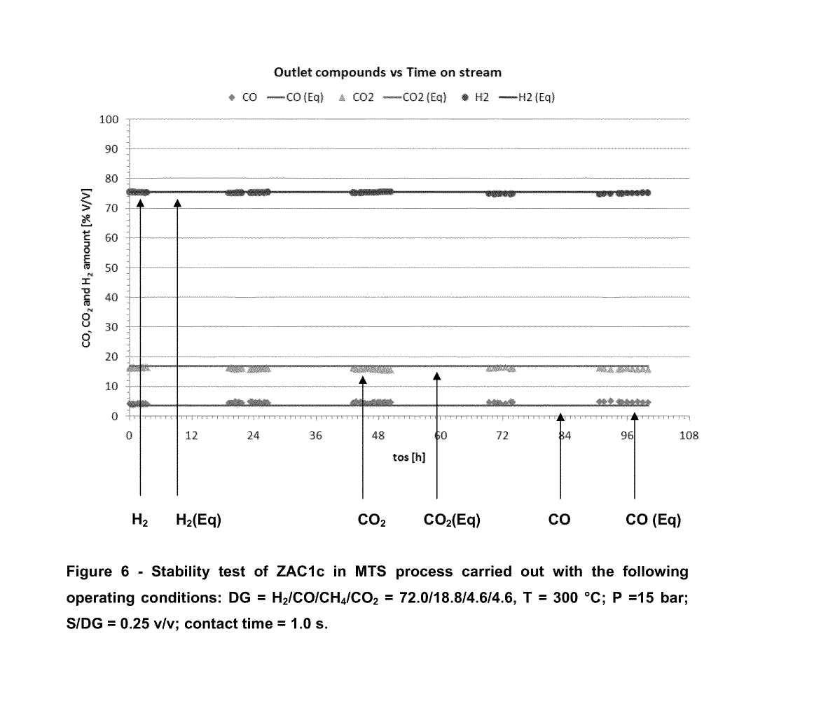

FIG. 6 shows the results using a ZAC1c sample. The long test demonstrates the stability of the performances of this catalyst with Time-on-stream (ToS) under pushed conditions in terms of the S/DG ratio (0.25 v/v) and resident time (1.0 sec). The outlet DG composition is close to equilibrium one and remained stable during the long test. The CO amount increased only of 0.7% after 100 h of ToS, evidencing a very stable behaviour of the ZAC1c catalyst also under pushed shift conditions.

E] Comparison Between the Catalysts According to the Invention and with ZA3K Catalyst

The activity and selectivity of the seven calcined HT catalysts in the WGS reaction carried out at medium temperature is studied. The following figures clearly show the best performance of Cu-containing catalysts derived from the calcination of carbonate-containing precursor, in terms of activity and selectivity, under typical industrial conditions, between 250° C. and 350° C.

As shown in FIG. 4, the sample without copper ZA3K shows a relatively good activity at high temperature, but the CO conversion is associated to a H₂ consumption, due to the significant presence of side-reactions with formation of oxygenated products (mainly methanol), attributable to its high basic character. Therefore, it seems not actually suitable as catalyst for the water gas shift process processes.

The catalysts according to the invention are obtained from the hydrotalcite structure containing Cu, Zn, Al and having carbonate or silicate as interlayer anions, after calcination and metal activation by reduction.

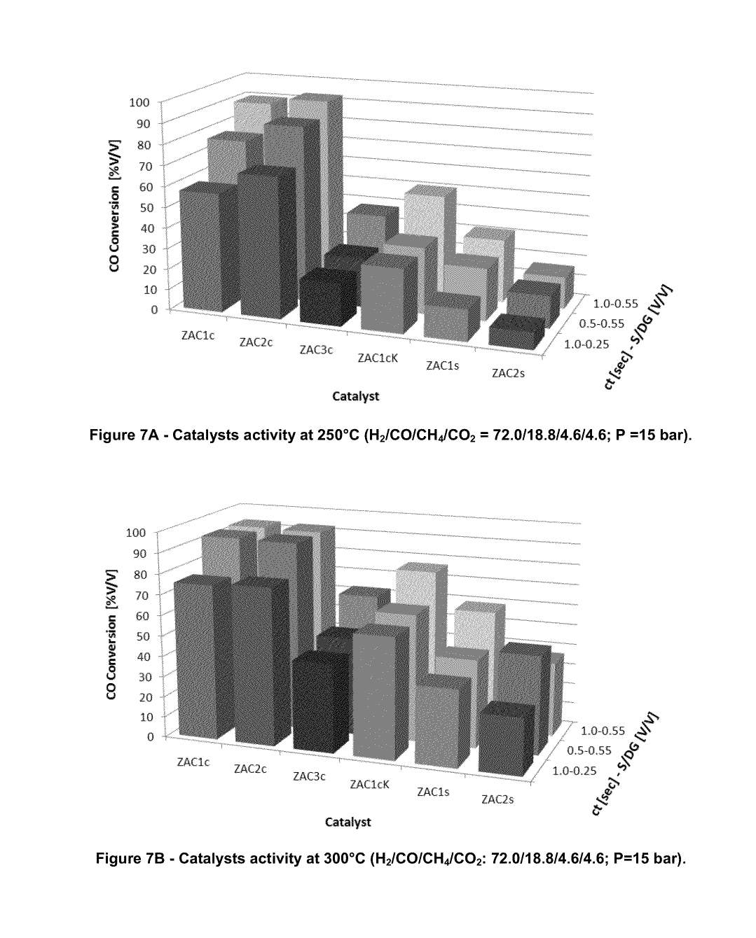

As shown in FIGS. 7A, 7B and 7C, The ZAC catalysts obtained from silicate-containing HT precursors are selective but poorly active, while the ZAC catalysts obtained from carbonate-containing HT precursor with carbonate as interlayer anion, are highly active and selective. All the tested Cu-based catalysts obtained from carbonate-containing HT precursor show very good performances in all the operating conditions, regardless of S/DG ratio and contact time values adopted, particularly when the Cu content is lower than 20 wt. %. At 250° C., a marked dependence on the operating conditions is observed, mainly related to the S/DG ratio. Both ZAC1c and ZAC2c are the most active catalysts, with values of CO conversion close to equilibrium ones, regardless of the S/DG ratio, when temperature is above 300° C.

Moreover, both catalysts also show the same decrease of BET surface area after reaction, although practically without sintering of Cu⁰ crystallites, justifying their stable behaviour with time-on-stream after 100 h.

In conclusion, even if ZA3K catalyst is able to partially convert CO but with a significant H₂ consumption due to side-reactions, the various ZAC catalysts which all contains copper, show significantly better performances, due to the promoting effect of this metal. The ZAC2c sample may be considered the best catalyst, suitable also for non-conventional values of the S/DG ratio. The very good performances in the MTS conditions, associated with a good thermal stability, justify the use of ZAC catalysts in the WGS processes, in order to minimize sintering phenomena and side-reactions with H₂ consumption.

F] Comparison with a Commercial Catalyst

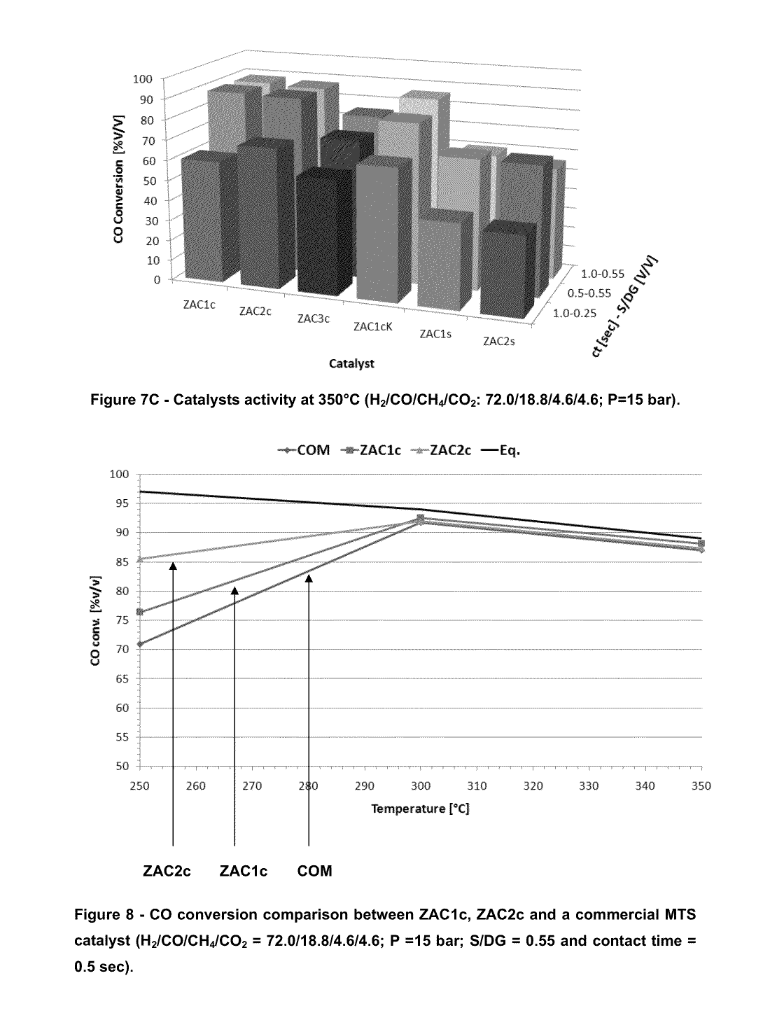

As shown on FIG. 8, the catalytic activities of catalysts according to the present invention were compared with a widely diffuse commercial catalyst, evidencing better performances, approaching the equilibrium values, for the best catalysts, obtained from carbonate-containing HT precursors, with Cu-contents lower than 20 wt. %. These catalysts show also a very good stability with time-on-stream.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary a range is expressed, it is to be understood that another embodiment is from the one.

Optional or optionally means that the subsequently described event or circumstances may or may not occur. The description includes instances where the event or circumstance occurs and instances where it does not occur.

Ranges may be expressed herein as from about one particular value, and/or to about another particular value. When such particular value and/or to the other particular value, along with all combinations within said range.

All references identified herein are each hereby incorporated by reference into this application in their entireties, as well as for the specific information for which each is cited.

1-12. (canceled)

13. A hydrotalcite-type compound of the formula (I):
[CuₓZn_{y}Al_{w}(OH)₂]^{(2x+2y+3w-2)+}(A²⁻)_{(2x+2y+3w-2)/2},kH₂O (I)
wherein:
(A²⁻) represents either a carbonate anion or a silicate anion,
x>0,
y>0,
w>0,
(x+y)=(1−w),
1<[(x+y)/w]<5, and
1/99≦x/y≦1/1.

14. The hydrotalcite-type compound of the formula (I), as claimed in claim 13, wherein the atomic ratio x/y≦1/2.

15. The hydrotalcite-type compound of the formula (I), as claimed in claim 13, wherein the atomic ratio x/y≦1/5.

16. The hydrotalcite-type compound of a formula (I), as claimed in claim 13, wherein the atomic ratio x/y≧1/10.

17. The hydrotalcite-type compound of a formula (I), as claimed in claim 13, wherein the atomic ratio [(x+y)/w] is higher or equal to 2.

18. The hydrotalcite-type compound of a formula (I), as claimed in claim 13, wherein the atomic ratio [(x+y)/w] is less or equal to 3.

19. A hydrotalcite-type compound of one of the following formulas:
[Cu_{0.075}Zn_{0.675}Al_{0.25}(OH)₂]^{0.25+}(CO₃²⁻)_{0.125}kH₂O
[Cu_{0.150}Zn_{0.600}Al_{0.25}(OH)₂]^{0.25+}(CO₃²⁻)_{0.125}kH₂O
[Cu_{0.066}Zn_{0.600}Al_{0.333}(OH)₂]^{0.25+}(CO₃²⁻)_{0.125}kH₂O
[Cu_{0.134}Zn_{0.532}Al_{0.333}(OH)₂]^{0.31+}(CO₃²⁻)_{0.155}kH₂O

20. A process for synthesizing the hydrotalcite-type compound of the formula (I) as claimed in claim 13, comprising the following steps:
a step A during which an aqueous solution containing together, aluminium nitrate, zinc nitrate and copper nitrate is prepared by mixing said nitrate salts in the appropriate proportions, with water;
a step B during which, said solution obtained at step A is mixed with an aqueous solution of hydrogenocarbonic acid sodium salt, the pH being maintained between 8 and 10, preferably around 9, to produce a precipitate; and
a step C during which, said precipitate obtained at step B, is isolated by filtration, washed and then dried to form the expected hydrotalcite-type compound of said formula (I).

21. The process as claimed in claim 20, further comprising the step of:
a step D during which said hydrotalcite-type compound of said formula (I), obtained at step C, is grinded to form a powder of particles of said hydrotalcite-type compound of said formula (I).

22. A compound obtained by a process comprising the following steps:
a step F during which a powder of the hydrotalcite-type compound of the formula (I) as claimed in claim 13 is calcined;
a step G during which the calcined powder obtained at step F is reduced with hydrogen, at a temperature of less than 230° C.

23. A process for performing a water gas shift reaction of a synthetic gas, the process comprising the steps of obtaining the compound as claim in claim 22; and using said compound as a catalyst for the water gas shift reaction of the synthesis gas.

24. A process for synthesizing methanol, the process comprising the steps of obtaining the compound as claim in claim 22; and using said compound as a catalyst for the synthesis of methanol.
